(19) United States
(12) Patent Application Publication
(10) Pub. No.: US 2011/0190683 A1
(43) Pub. Date: Aug. 4, 2011

(54) EXPANDABLE AND COLLAPSIBLE MEDICAL DEVICE

(75) Inventors: Barry N. Gellman, North Easton, MA (US); J. Scott Richardson, Wilmington, MA (US); Kurt A. Dasse, Needham, MA (US)

(73) Assignee: LEVITRONIX LLC, Waltham, MA (US)

(21) Appl. No.: 13/018,938

(22) Filed: Feb. 1, 2011

Related U.S. Application Data
(60) Provisional application No. 61/300,603, filed on Feb. 2, 2010.

(51) Int. Cl. A61M 1/14; A61M 29/02

(52) U.S. Cl. 604/6.16; 606/191

(57) ABSTRACT

A catheter (or cannula) is small enough in diameter to be placed minimally invasively into the body of a patient but also can be expanded post-placement to provide a larger-diameter placed catheter that supports fluid flow at a rate higher than is possible through the pre-placement reduced-diameter catheter. The expandable catheter is constructed using one or more shape memory polymers and can include one or more stent-like sections and/or at least part of the cannula formed as folded lobes. Each of the stent-like sections is configured to enhance the flexibility of the section as compared to the other parts of the catheter and thus allow the section to accommodate tight bends and turns when inserted into the body of the patient. The folded lobes are axial folds that constitute at least one section of the cannula, and they unfold upon expansion of the catheter to create a large cross-sectional shape.

### CROSS-REFERENCE TO RELATED CASES

This claims the benefit of and priority to Provisional U.S. Patent Application No. 61/300,603 which was filed on Feb. 2, 2010. The entirety of this provisional patent application is incorporated herein by reference.

Also incorporated herein by reference in its entirety is commonly-owned U.S. Patent Application Publication No. US 2007/0233041 A1 which was published on Oct. 4, 2007. And finally the entirety of commonly-owned U.S. Patent Application Publication No. US 2007/0197856 A1, which was published on Aug. 23, 2007, also is incorporated herein by reference.

### TECHNICAL FIELD

The invention relates to medical devices that can be expanded and collapsed and, more particularly, to catheters or cannulae that expand after placement within the body of a patient to allow increased fluid flow.

### BACKGROUND INFORMATION

It is known that one or more catheters or cannulae may be used to access the heart and other major blood vessels of the human body to achieve the transport of blood and/or one or more other fluids into and/or out of the body. Cardiac cannulae provide the patient interface to extracorporeal blood circuits, and these cannulae typically are placed by cardiac surgeons or cardiologists under direct vision into major blood vessels and/or the heart for the purpose of providing one or more fluid conduits to and from the extracorporeal circuits.

While the words catheter and cannula may be used interchangeably herein, it is noted that cardiologists typically use “catheter” whereas cardiac surgeons typically use “cannula”.

The most common techniques and devices used in cardiac surgery centers for postcardiotomy support include extracorporeal life support (ECLS), extracorporeal membrane oxygenation (ECMO), extracorporeal CO₂ (i.e., carbon dioxide) removal (ECCO₂—R), and ventricular assist devices (VAD). Poor ventricular function may be diagnosed preoperatively or may have resulted from myocardial insult during surgery (e.g., inadequate perfusion, cross-clamping for extended periods of time limiting reperfusion, injury, etc.). Reduced cardiac output will affect other organs due to low blood pressure and low blood flow. Allowing the myocardium to rest for a certain period of time may allow recovery, but if sufficient recovery does not occur the patient may require long-term cardiac support. Patients who cannot be weaned from cardiopulmonary bypass and possess isolated ventricular dysfunction may be candidates for a VAD. Two-pump circuits are required to support a patient with a biventricular assist device (BiVAD). When pulmonary dysfunction occurs with or without cardiac dysfunction, the patient may be a candidate for ECLS, ECMO, or ECCO₂—R support.

A cardiac cannula can provide access via the vasculature through major vessels such as the right atrium (RA), left atrium (LA), left ventricular apex (LVA), femoral artery (FA), femoral vein (FV), superior vena cava (SVC), inferior vena cava (IVC), internal jugular vein (IJV), carotid artery (CA), subclavian artery (SA), the aorta, and other major chambers or blood vessels. Two lumens typically are required in the extracorporeal circuit, where one of the lumens is used for blood inflow from the patient's body and the other one of the lumens is used for blood return into the patient's body. These lumens can be the two separate lumens that are integrated into a dual-lumen cannula (also known as a dual-lumen catheter), or the two lumens can be represented by a one cannula with a single lumen and a second cannula with a single lumen. When a dual-lumen cannula is employed, only one device needs to be placed into the patient to establish the inflow and outflow channels. This is in contrast to the use of two separate single-lumen catheters. When using two cannulae, one of the single-lumen catheters needs to be directed from the patient to the blood pump, and the other single-lumen catheter needs to be placed from the pump to the patient.

In open chest procedures, a single-lumen inlet (drainage) cannula (which provides blood to the extracorporeal circuit from inside the patient's body) can be passed from the ventricle or atrium through the subcutaneous plane to the percutaneous access site on the patient's body and externalized. Alternatively, a single-lumen outlet or return cannula (which takes blood from the extracorporeal circuit and conveys it into the patient's body) can be passed percutaneously through a subcutaneous tunnel to the arch of the ascending aorta (or another vessel such as the subclavian artery) and into the lumen of the vessel to return the blood to the patient. Both of the percutaneous access and exit sites are often located ipsilaterally on the left abdominal wall for a left ventricular assist device (LVAD) and in the medial anterior position. The location is often ipsilateral on the right abdominal wall for a right ventricular assist device (RVAD) and in the medial anterior position. Cannulae placed within the thorax are typically secured in place to prevent accidental dislodgement which could result in a catastrophic condition. Purse-string sutures and optional stabilizer grommets can used to provide anatomic fixation until tissue healing occurs. As such, cannulae removal and/or exchange requires a second surgery under direct visualization. The open chest wound is closed after circulatory support has been terminated.

Non-open chest procedures are less invasive and generally preferred if an open chest procedure can be avoided. Inlet and return catheters also need to be placed in non-open chest procedures, but in non-open chest procedures the diameter of each of the placed catheters typically must be ideally smaller to allow placement by navigating the catheter into the patient's blood vessel(s). Typically, a guidewire is placed first and the catheter rides over the guidewire to be placed within the patient's body, but even with the aid of a guidewire in placing a catheter in a minimally invasive procedure, the catheter typically must make sharp turns within the vasculature and needs to be of a relatively small diameter for ease-of-placement purposes. Smaller diameter catheters are not able to deliver the flow rates of larger diameter catheters.

Vascular stents also are currently available. The purpose of a vascular stent is to hold open a blood vessel, such as a coronary artery, by pushing against the inner wall of the blood vessel. A vascular stent can be made of metal, and it can be made to elute one or more drugs into the patient. Regardless of the type or design of a vascular stent, a stent is not intended, designed, or used for transporting one or more fluids (such as blood) into or out of the body of a patient.

Shape memory polymers are known. A shape memory polymer is a polymeric material that is able to reform from a deformed, temporary shape to an original, permanent shape by an external stimulus or trigger such as a temperature change.

### SUMMARY OF THE INVENTION

The invention generally relates to a catheter (or cannula) that is small enough in diameter to be placed minimally invasively into the body of a patient, that can also be expanded after placement to provide a larger-diameter form that supports fluid flow at a sufficiently high rate even though the pre-placement smaller-diameter shape could not support such a high flow rate. In accordance with the invention, such an expandable catheter is constructed using one or more shape memory polymers. Once expanded, the catheter can be returned to its collapsed state. The expanded and collapsed states can be achieved any number of times as desired or needed by the operator. The expandable/collapsible catheter according to the invention can include one or more stent-like sections where each of these sections includes a plurality of holes, ports, or other openings to enhance the flexibility of the section to accommodate tight bends and turns such as those present in a path within the vasculature of a patient's body when trying to locate the distal tip of the catheter within an artery or vein of the patient. In addition to or instead of the one or more stent-like flexible sections, the shape memory polymer (SMP) expandable/collapsible catheter of the invention can have one or more sections with axial folds that open or unfold upon expansion to create a large cross-sectional circular shape. That is, instead of expanding after placement from a smaller circular diameter to a larger circular diameter, the SMP expandable catheter can have one or more sections with multi-lobed axial folds that allow the smallest possible collapsed diameter for insertion into the body and the largest possible expanded circular diameter after placement into the patient.

In one aspect, the invention involves a catheter with a collapsed diameter sufficient to allow placement of the catheter over a guidewire and into a body of a patient. The catheter is expandable in diameter after placement in the body of the patient to support fluid flow at a predetermined rate higher than a rate possible with the collapsed diameter. The catheter comprises an elongated member including a tubular wall defining at least one lumen extending therethrough. The elongated member comprises a shape memory polymer which allows at least a portion of the elongated member to expand its diameter beyond its collapsed diameter when exposed to at least one triggering stimulus (such as, for example, temperature, electricity, or light). In addition, at least one section of the elongated member comprises a plurality of holes in the tubular wall to make that section more flexible and bendable than other parts of the elongated member without such holes.

In another aspect, the invention relates to a medical device for insertion into a blood vessel of a patient and for carrying one or more fluids into and/or out of the patient's body. The medical device comprises a hub for placement outside the patient's body and an elongated member extending from the hub and defining at least one passageway. At least one portion of the elongated member comprises a shape memory polymer, and at least this portion of the elongated member is designed to be placed within the patient's blood vessel when the elongated member is in a compressed state. In the compressed state, at least one passageway is sized to receive a guidewire but not to carry fluid at a high predetermined rate into and/or out of the patient's body. At least one portion of the elongated member is configured to change to an expanded state after placement within the patient's blood vessel in order to be able to carry fluid at the sufficiently high predetermined rate. Finally, at least one section of one portion of the elongated member is more flexible than the remaining section of the elongated member.

Embodiments according to this aspect of the invention can include various features. For example, one or more sections of one or more portions of the elongated member can have a plurality of holes in a wall of the elongated member to make that section more flexible and bendable than other parts of the elongated member without such holes. The elongated member can further comprise one or more sections with a plurality of axial folds that unfold when the elongated member is in the expanded state. The blood vessel can be a vein or an artery, and blood or another fluid can be carried into and/or out of the patient's body. The elongated member can define two passageways, and thus the medical device would be a dual-lumen catheter. The shape memory polymer can be activated by heat, light, or electricity, for example, in order to change the elongated member from the compressed state to the expanded state. The elongated member can be configured such that it does not exert a pushing force on the interior wall of the patient's blood vessel when placed within the patient's blood vessel and in the expanded state, and the placed/expanded elongated member thus would not block the flow of blood through the blood vessel but instead would allow blood to flow between the interior wall of the patient's blood vessel and the exterior surface of the elongated member. The hub of the medical device can include connectors for connecting to an extracorporeal support system such as an extracorporeal blood circuit.

In yet another aspect, the invention involves a medical device for insertion into a blood vessel of a patient and for carrying one or more fluids into and/or out of the patient's body. The medical device comprises an elongated member defining at one or more passageways. At least one portion of the elongated member comprises a shape memory polymer and can be placed within the patient's blood vessel when the elongated member is in a compressed state in which one or more passageways are sized to receive a guidewire but not to carry fluid at a sufficiently high predetermined rate into and/or out of the patient's body. The one or more portions of the elongated member are configured to change to an expanded state after placement within the patient's blood vessel in order to be able to carry fluid at the sufficiently high predetermined rate. At least one section of the portion of the elongated member comprises a plurality of axial folds that unfold when the elongated member is in the expanded state.

Embodiments according to this other aspect of the invention can include various features. For example, at least one other section of the portion of the elongated member can comprise a plurality of holes in a wall of the elongated member to make that section more flexible and bendable than other parts of the elongated member without such holes. The blood vessel can be a vein or an artery, and at least blood can be carried into and/or out of the patient's body. The elongated member can define two passageways such that the medical device is a dual-lumen catheter, and these two lumens can be oriented coaxially or side-by-side, for example. The shape memory polymer can be activated by heat, light, or electricity, for example, in order to change from the compressed state to the expanded state. The elongated member can be configured such that it does not exert a pushing force on the interior wall of the patient's blood vessel when placed within the patient's blood vessel and in the expanded state, and the placed/expanded elongated member thus would not block the flow of blood through the blood vessel but instead would allow blood to flow between the interior wall of the patient's blood vessel and the exterior surface of the elongated member. The elongated member can extend from a hub of the medical device, and the hub can include connectors for connecting to an extracorporeal support system.

Objects, advantages, and details of the invention herein disclosed will become apparent through reference to the following description, the accompanying drawings, and the claims. The various disclosed embodiments as well as each of the various features of those embodiments are not mutually exclusive and can exist in various combinations and permutations whether or not expressly pointed out in the following description or the accompanying drawings.

### BRIEF DESCRIPTION OF THE DRAWINGS

In the drawings, like structures are referenced by the same or similar reference numbers throughout the various views. The illustrations in the drawings are not necessarily drawn to scale, the emphasis instead being placed generally on illustrating the principles of the invention and the disclosed embodiments.

FIG. 1A is a side view of an embodiment of a medical device according to the invention, with a cannula of the device in its collapsed state such that an inside diameter and an outside diameter of the cannula are minimized but sufficient to receive a guidewire, and with breakaway lines to indicate that the entire length of the cannula is not shown.

FIG. 1B shows an enlarged view of a distal section of the collapsed cannula of the device of FIG. 1A.

FIG. 1C is a cross-sectional view at any point along the collapsed cannula of the device of FIGS. 1A and 1B, with a minimized-diameter lumen shown that extends through the device when its cannula is in the collapsed state.

FIG. 2A depicts the medical device of FIG. 1A but with a different type of hub configuration and also with the cannula in its expanded state whereby the inside diameter and the outside diameter of the cannula are maximized to allow a higher flow rate through the increased-diameter lumen than is possible through the minimized-diameter lumen shown in FIG. 1C.

FIG. 2B shows an enlarged view of a portion of the distal section of the expanded cannula of the device of FIG. 2A with holes in this expanded distal section that make it more flexible and bendable than other parts of the cannula without such holes.

FIG. 2C is a cross-sectional view at any point along the expanded cannula of the device of FIGS. 2A and 2B, with the maximized-diameter lumen shown that extends through the device when its cannula is in the expanded state.

FIG. 3A is a view of the medical device of FIG. 2A with the cannula expanded but with the distal section of the expanded cannula in a particular curved configuration that it assumes due to the transition from the collapsed state to the expanded state.

FIG. 3B shows an enlarged view of a portion of the distal section of the expanded cannula of the device of FIG. 3A with holes in this expanded distal section that make it more flexible and bendable than other parts of the cannula without such holes.

FIG. 4 depicts the medical device of FIG. 1A but without the breakaway lines in the cannula's length and also with two or more distinct and separate sections of the collapsed cannula as opposed to just the one distal section shown in FIGS. 1A and 1B.

FIG. 5 depicts the medical device of FIG. 1A but with only a portion of the collapsed cannula shown and also with a portion of a peel sheath shown disposed over the collapsed cannula for the purpose of delaying the expansion of the cannula into its expanded state until placement of a distal end of the collapsed cannula at a particular location within a body of a patient can be verified.

FIG. 6A shows the distal section of the expanded cannula of the device of FIG. 2A but with holes of a different shape and pattern as compared to the holes in the expanded distal section shown in FIG. 2B.

FIG. 6B shows the same distal section of the expanded cannula as in FIG. 6A but with a flexible thin-walled sheath disposed both inside and outside of at least this distal section of the expanded cannula to cover the holes and thus allow the same flexibility and bendability of this distal section while maintaining a continuous fluid path within the expanded-diameter lumen of the device.

FIG. 6C shows an angled hole made in the wall of the cannula of a medical device according to the invention in order to achieve a distal section of the expanded cannula (similar to that shown in FIG. 6A) with such holes.

FIG. 6D shows a protruding hole formed in the wall of the cannula of a medical device according to the invention in order to achieve a distal section of the expanded cannula (similar to that shown in FIG. 6A) with such holes.

FIGS. 7A, 7B, and 7C depict cross-sectional views of a multi-lobed cannula embodiment of a medical device according to the invention, where the device can be any of the devices shown in FIGS. 1A, 2A, 3A, 4, and 5, and where FIGS. 7A and 7C show the cannula in its fully collapsed and fully expanded states, respectively, and FIG. 7B shows the cannula in transition from the collapsed state to the expanded state. Axial folds of this multi-lobed cannula allow folding of the wall of the cannula as it reduces down to its collapsed state and unfolding of the wall of the cannula as it opens up to its expanded state, and these folds can be created by rolling the body of cannula.

FIG. 8 depicts the medical device of FIG. 1A but with the device having the distal section of its cannula configured to be mechanically expanded by a balloon as opposed to being made of a shape memory polymer that is expanded by a trigger such as an increase in temperature.

FIG. 9A is a cross-sectional view of an embodiment of a medical device according to the invention, with breakaway lines along the length of a dual-lumen cannula of the device to indicate that the entire length of the dual-lumen cannula is not shown.

FIGS. 9B and 9C show two possible cross-sectional configurations for the two lumens of the dual-lumen device of FIG. 9A, where each of these configurations has an optional smaller center lumen for receiving a guidewire.

FIG. 9D is a cross-sectional view of a coaxial arrangement of two lumens of a dual-lumen cannula of a medical device according to the invention.

FIG. 9E is a cross-sectional view of a multi-lumen cannula of a medical device according to the invention, where three separate lumens are shown in this particular multi-lumen embodiment and also along with an optional center lumen for receiving a guidewire.

FIG. 10A is a cross-sectional view of an embodiment of a medical device according to the invention, with a dual-lumen cannula of the device in its collapsed state such that an inside diameter and an outside diameter of an outer of the two eccentric lumens are minimized and such that the outer lumen prevents flow therethrough.

FIG. 10B shows the device of FIG. 10A but with the outer eccentric lumen in its expanded state to allow flow therethrough.

FIG. 11A is a cross-sectional view of an embodiment of a medical device according to the invention, with a dual-lumen cannula of the device in its collapsed state such that an inside diameter and an outside diameter of both the inner and outer coaxial lumens are minimized and such that the outer lumen prevents flow therethrough.

FIG. 11B shows the device of FIG. 11A but with the outer coaxial lumen in its expanded state to allow flow therethrough.

FIG. 12 is a cross-sectional view of a collapsed-state/non-expanded cannula (such as the cannula shown in side view in FIG. 1A) with a distal end portion that tapers down and with an introducer needle extending through the lumen of the cannula.

FIG. 13 is a cross-sectional view of a collapsed-state/non-expanded cannula with a sharpened and angled distal end to allow it to puncture the skin or other tissue of a patient without the need for a separate introducer needle.

FIG. 14 is a cross-sectional view of a collapsed-state/non-expanded cannula (such as the cannula shown in side view in FIG. 1A) disposed within a lumen of an introducer needle.

FIG. 15A is a cross-sectional view of a cannula of a medical device according to the invention, where the device can be any of the devices shown in FIGS. 1A, 2A, 3A, 4, 5, 7A-7C, 9A, 10A, 10B, 11A, 11B, and 12-14, and where at least a portion of the cannula includes wires embedded in the wall of the cannula for electrical activation of a shape memory polymer to transition the cannula between its collapsed and expanded states.

FIG. 15B is a cross-sectional view of a cannula of a medical device according to the invention, where the device can be any of the devices shown in FIGS. 1A, 2A, 3A, 4, 5, 7A-7C, 9A, 10A, 10B, 11A, 11B, and 12-14, and where at least a portion of the cannula includes a shape memory polymer containing a plurality of micro-particles and/or nano-particles which can be excited by light (from, for example, a laser light source) or by induction (whether electromagnetic, electrostatic, or magnetic) in order to transition the cannula between its collapsed and expanded states.

### DESCRIPTION

A single-lumen device according to the invention can be positioned in a large chamber or vessel to enable aspiration or fluid discharge near the distal end of the device. Alternatively, multiple hole locations may be included some distance apart along the device's shaft to result in drainage or aspiration from another anatomical location (e.g., atrium and kidney). A single-lumen device of the invention can be positioned through a tortuous distal path for distal fluid aspiration or fluid discharge. A multi-lumen device according to the invention can be capable of performing fluid aspiration and/or re-infusion from and/or to multiple sites within the patient's body simultaneously. Medical devices according to the invention, whether referred to as cannulae or catheters, can be utilized individually or together to transport venous or arterial blood from/to the vasculature and an extracorporeal circuit and/or extracorporeal device such as a pump, oxygenator, heat exchanger, etc. The vascular access may be a single site vena-puncture or multiple site vena-puncture. Vascular access may be attained through visual means, palpated means, or placed with the aid of imaging such as fluoroscopy, ultrasound, or other visualization. It is intended to provide vascular access through percutaneous access (through the skin) either directly with the cannula or with the aid of a guide wire, dilator, or the Seldinger technique or a modified Seldinger technique. Alternatively, a minor vascular cut-down may be necessary to avoid excessive vascular injury during placement or cannula expansion. For major flow requirements such as utilized in ECLS, ECMO, ECCOR, or VAD utilization, the distal cannula(e) tips (that is, the ends closest to the heart) will most likely reside within a major vessel such as the Inferior or Superior Vena Cava, Right Atrium, or Right Ventricle on the venous side. Return blood may be delivered to the same vessel or vessels including the Pulmonary Artery for right-sided veno-venous or veno-arterial support, and to the left side of the heart (left ventricle, left atrium or aorta). Access to these vessels may be percutaneously attained through feeder vessels such as the femoral artery, femoral vein, carotid artery, subclavian artery and other major arteries.

The invention relates to a medical device with a self-expanding catheter or cannula that utilizes one or more shape memory polymer (SMP) materials. Medical devices according to the invention provide a conduit (also referred to as a passageway or a lumen) for the transport of one or more fluids into and/or out of the body of a patient through one or more blood vessels of the patient. One device according to the invention (whether referred to as a catheter or a cannula) includes a hub and an elongated member extending from the hub. The entire elongated member, or more typically some portion of it, gets placed within a blood vessel of the patient, and at least a portion of the elongated member that gets placed within the patient's body is shape-changing due to being formed of an SMP. The elongated member is the cannula. At least a portion of the elongated member can change from a compressed or collapsed state into an expanded state. The geometry of the shape memory polymer may be adjusted to resist compression by soft tissue at the skin penetration site and along the subcutaneous tunnel, and may be adjusted to be more flexible along its length for the portion within the blood vessel. The hoop strength of the diameter of the catheter may be increased by increasing the wall thickness locally for the device. The cannula is inserted into a blood vessel in the collapsed state when it has a smaller diameter for ease of introduction and positioning, and it expands to a larger bore conduit for allowing fluid to flow at a sufficiently high rate into and/or out of the body. In the collapsed state, it can receive and ride over a guidewire, but the smaller diameter of the elongated section in the collapsed state is not intended to allow fluid to flow through the elongated section at a sufficiently high rate. The shape change capability results from forming at least some of the elongated member of one or more SMPs, and the shape change can be triggered by a change in temperature, application of electrical current, light, moisture, or some other triggering stimulus. The medical device can be configured to shape change by a combination of more than one triggering stimuli. And the medical device can be collapsed after it is expanded and then expanded again. The medical device can be expanded and collapsed any number of times, as desired or needed by the operator.

A medical device according to the invention can include at least: (1) a shape memory tube (with a variety of possible distal tip, body, and cross-sectional configurations) that will be positioned within a chamber or blood vessel of the patient's internal body, and (2) an external hub component for extracorporeal connection to a pump device or extracorporeal blood circuit components. The cannula tube is configured such that the maximum diameter of the tube is realized at or about normothermic temperature (that is, from 34° C. to 37° C.), such that the phase change of the cannula will occur within the patient's body after placement. The recovery of the polymer phase change (that is, the expansion to the expanded state from the collapsed state due to the SMP properties) may be triggered by temperature conduction through the SMP material(s), and this conduction of temperature can occur over time such as while the cannula is being placed within the body of the patient. The device can be configured such that its placement within the body of the patient is completed before the temperature change/conduction causes the SMP cannula to reach its maximum expanded state. The recovery of the polymer phase change may be inhibited (to, for example, provide an operator with more time to place and/or reposition the device within the body of the patient) by containing at least the cannula tube portion of the medical device within a sheath. As the cannula is advanced into the patient's body, it is contained within the sheath which is advanced simultaneously with the cannula into the patient's body. The sheath is then removed by the operator from the patient's body. A known peelable introducer sheath can be used for this purpose. The recovery of the polymer phase change thus may be initiated by a temperature increase due to moving the SMP cannula from the atmosphere (which may be about 20° C., for example) to the higher temperature inside the body of a patient. This recovery or expansion may instead be triggered by something other than a temperature increase, such as electrical inducement from a DC supply. Other change stimuli are possible as well, such as light, and combinations of different change stimuli could be employed to move the cannula from its collapsed state to its expanded state. The particular SMP(s) used to form the cannula will dictate what triggers the change in state of the cannula from collapsed to expanded and also back again from expanded to collapsed.

Once expanded within the body of a patient, the SMP cannula of the invention could be returned to its collapsed state while the cannula is in place within the body of the patient. An operator may want to return the cannula to its collapsed state to, for example, temporarily reduce or even stop fluid flow through the cannula until the SMP is activated again to increase the size and again permit fluid flow. A reduced-size/collapsed cannula also would be easier to remove from the body of the patient.

Medical devices according to the invention (whether of the single-lumen, dual-lumen, or multi-lumen type) can be configured and constructed as described herein. It is noted that the length of any particular cannula or catheter of a medical device according to the invention will be such that a distal tip of the catheter is located within the desired chamber and/or blood vessel within the human body once placement of the catheter within the body is complete. The length of a particular catheter typically will be selected and set on this basis.

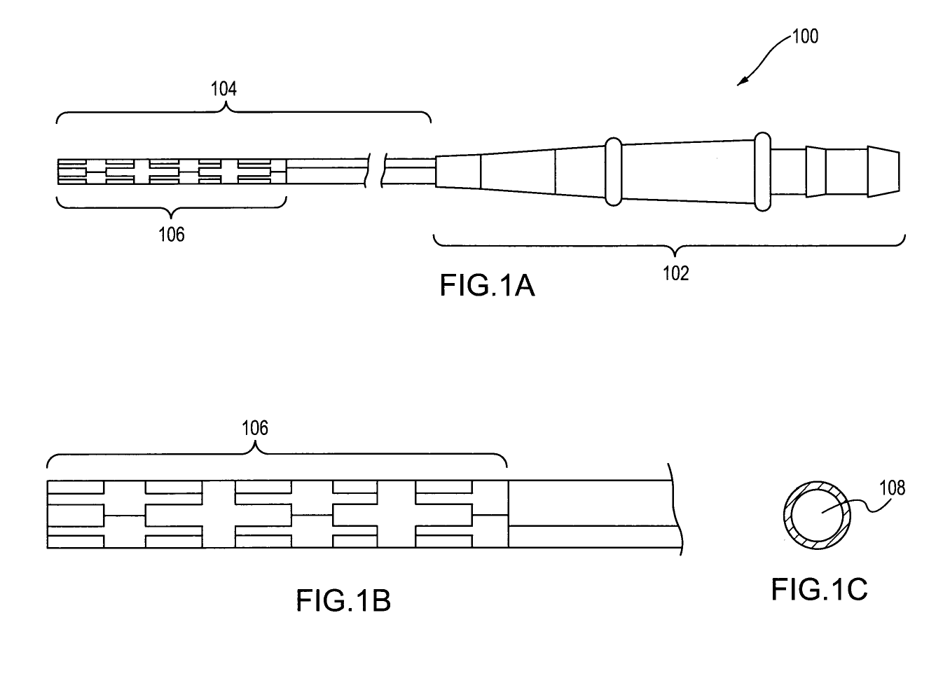

Referring to FIGS. 1A-1C, a medical device 100 according to the invention includes a hub 102 and a straight cannula or catheter 104 in its collapsed state. The hub 102 is hermetic to the cannula 104. The cannula 104 includes at least one section 106 that is distinct from at least one other section of the cannula 104. In the embodiment of the medical device according to the invention that is depicted in FIGS. 1A and 1B, one distinct section is shown. It is the collapsed distal section 106, and it is located at the distal end portion of the cannula 104 in this particular disclosed embodiment. All or most of the entire length of the cannula 104 is constructed, according to the invention, of one or more shape memory polymer (SMP) materials. An alternative is to have one or more portions or sections of the cannula 104 formed of one or more SMPs, but in a preferred embodiment all or most of the length of the cannula comprises one or more SMPs. Forming the cannula 104 of SMP(s) allows the cannula 104 to be advanced into the body of the patient in its small-diameter (that is, compressed or collapsed) state. The cannula 104 of the device 100, including its distal section 106, is in the compressed state, and in this state the cannula 104 has a through-lumen 108 with a diameter sized to receive a guidewire (e.g., 0.035″-0.038″ diameter). The device 100 would be advanced into the body of the patient over the guidewire after the guidewire is placed by an operator into the patient's body. Once an operator has finished positioning the cannula 104 inside the body of the patient, regardless of whether a guidewire is used or not, the cannula can be transitioned to its full expanded state. A clamp or valve can be utilized, either contained within the hub 102 or on perfusion tubing that connects to the hub 102, to halt the flow of fluid through the lumen 108 until necessary to engage the extracorporeal circuit to which the medical device 100 is connected. While the collapsed-state cannula 104 of the device 100 is shown and described as an elongated tubular member with a circular cross-sectional shape, it is noted that other cross-sectional shapes of the cannula 104 are possible such as elliptical, square, etc.

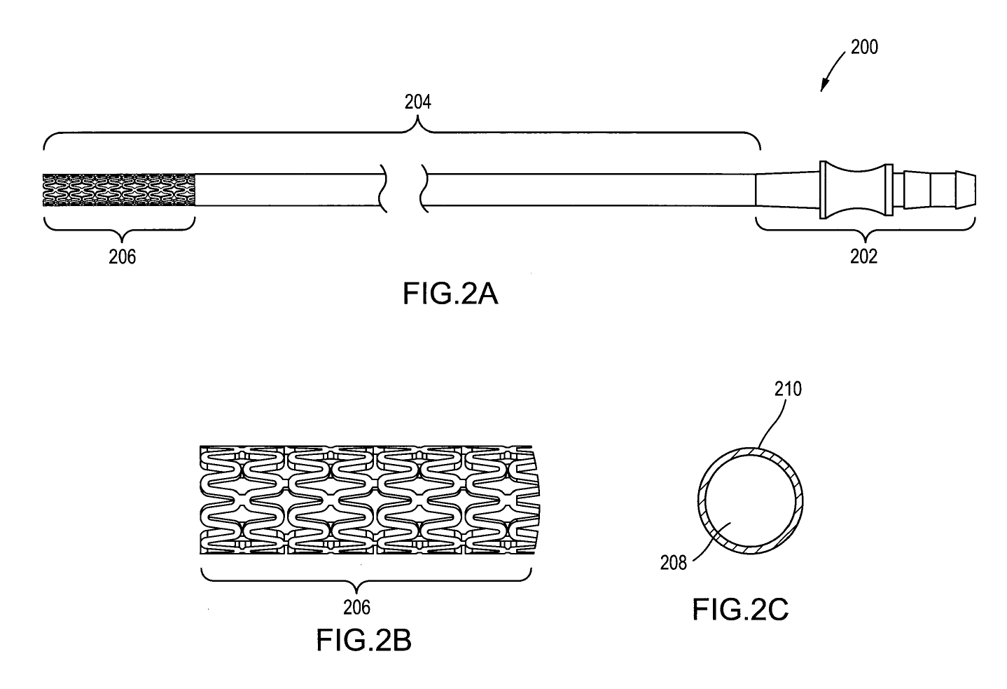

Referring to FIGS. 2A-2C, the expansion of the diameter of the straight cannula 204 can be accomplished by the effect of temperature, electrical current, light, and/or one or more other triggering stimuli of the SMP material(s). The cannula 204 as well as any drainage hole(s) in it, such as those in the expanded distal section 206, will expand to their maximum diameter after placement, and this results in the maximum-diameter through-lumen 208 being able to successfully transferring blood (and/or one or more other fluids) at a sufficiently high rate as compared to the lower rate possible through the minimum-diameter lumen 108 of the collapsed-state cannula 104. The cross-sectional size of the expanded-state lumen 208 will vary depending upon vessel placement size and fluid flow requirements. For example, for adult cardiopulmonary bypass, an acceptable size is 18 F to 25 F in order to be able to achieve a sufficiently high fluid flow rate through the lumen 208, although longer cannula have a greater pressure drop over their length which reduces flow and so length and diameter have to be considered in order to arrive at a cannula the provides a sufficient flow rate when the cannula is expanded. Pressure-flow curves are generated to identify the maximum permitted blood flow rate while not exceeding a negative 50 or negative 100 mmHg (depending which study). Above this limit, the vacuum will draw gas out of solution which should be avoided. In V-A ECMO support, higher flow rates are required of the cannula (up to total support of 6 LPM) as the lungs are being bypassed. In V-V ECMO support, much less flow can be utilized as it is an “assist” device.

The outer surface 210 of the wall of the expanded-state cannula 204 need not, and preferably does not, touch the inner wall of the vessel of the patient into which the cannula 204 has been placed. The expanded-state cannula 204 should be resistant to collapse under negative pressure imparted from the suction side of an extracorporeal pump. The expanded-state lumen 208 must generally maintain its size and shape in the face of forces imparted by muscles and the vessel wall of the patient through which the cannula 204 extends. If need be, the resistance to compression for the SMP can be adjusted for sections that will remain in the soft tissue to avoid collapse. While the expanded-state cannula 204 of the device 200 is shown and described as an elongated tubular member with a circular cross-sectional shape, it is noted that other cross-sectional shapes of the cannula 204 are possible such as elliptical, square, etc. With its plurality of drainage holes, the expanded-state distal section 206 of the cannula 204 has a stent-like configuration.

It is noted that in some embodiments the wall of the expanded cannula 204 is thinner than the wall of the collapsed cannula 104. This is because the SMP shape change causes the wall thickness of the cannula to decrease as the cannula moves from its collapsed state to its expanded state. However, in the multi-lobed cannula embodiment described herein (with reference, for example, to FIGS. 7A-7C), the SMP shape change is just opening or unfolding the folded lobes that form the cannula wall (or some portion or portions of it) and does not involve any thinning of the cannula wall when going from the collapsed or folded state to the expanded or unfolded state.

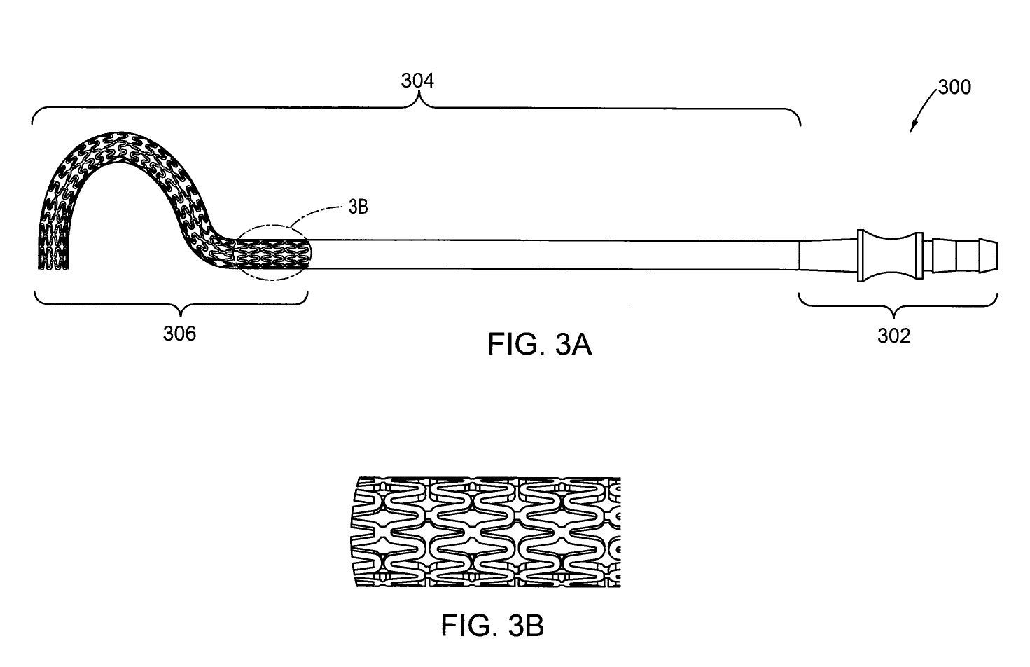

It is also noted that the expandable cannula may expand to multiple diameter segments and transitions along the length of the cannula body. That is, in its expanded state, the cannula may have different diameters at different points along its length, even though the embodiments shown in FIGS. 2A, 2C, and 3A, for example, do not indicate this multiple-diameter cannula possibility. The SMP catheter can be have graded resistance along at least a portion of the length of the catheter in order to minimize structural compression when the catheter is placed in soft tissue of the patient.

Instead of the straight cannula shown in FIGS. 1A-1C and 2A-2C, a medical device 200 of the invention can have a formed or shaped cannula 304 as shown in FIGS. 3A and 3B. This expanded-state cannula 304 also has a stent-like configuration with the plurality of holes in its distal section 306, but when the SMP is activated by a trigger the cannula 304 expands and assumes a pre-set shape such as the shape shown in FIG. 3A. In this particular embodiment, the expanded distal section 306 has a length of 4 inches and is designed and shaped to access the pulmonary artery of a patient from the patient's inferior vena cava. In addition to assuming the disclosed shape, the stent-like configuration of the expanded distal section 306, with its multiple holes, gives the distal section 306 (and, similarly, the distal section 206 of FIG. 2A) flexibility and the ability to bend without kinking. The solid, no-hole portions of the cannula 304 are prone to kinking when bent and are not as flexible or bendable as the distal section(s) 306 (or as the distal sections 206 and 106 of FIGS. 2A and 1A, respectively).

Referring still to FIGS. 3A and 3B, the non-straight cannula 304 can assume its shape as it expands to its expanded state from its collapsed state, as indicated above, or alternatively this cannula 304 can be pre-shaped by the fabrication process into a variety of preset configurations selected prior to insertion into a patient's body. For example, to place the distal tip of a cannula into the pulmonary artery of a patient, an operator can select a pre-shaped cannula to match the target anatomy and thus most easily make the required sharp bend to pass the cannula through the right atrium, advance it through the tricuspid valve to the right ventricle, make another sharp bend to advance it through the pulmonary valve, and end up with the distal tip of the cannula residing within the pulmonary artery. The cannula can be advanced by the operator by feel or under fluoroscopy or ultrasound or else following a Swan-Ganz catheter (which typically requires a slightly larger collapsed inside diameter of the cannula's lumen in order to fit the catheter). The cannula can be pre-shaped into any of a variety of single- or multi-bend configurations that are two-dimensional or three-dimensional.

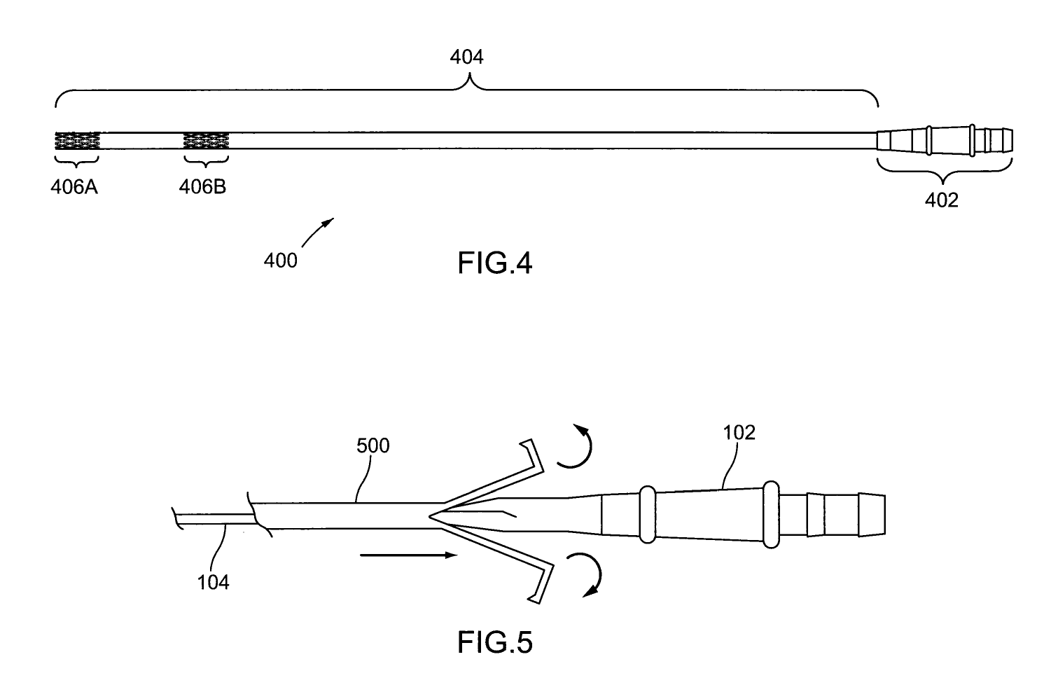

Referring now to FIG. 4, instead of a single distal section (106, 206, 306), a medical device 400 according to the invention can have a catheter 404 with two flexible distal sections 406A, 406B. More than two such sections are possible, but are not shown. Employing a cannula with two flexible sections such as depicted in FIG. 4 can allow an operator to place the cannula in the body of a patient and drain blood simultaneously from both the superior/inferior vena cava junction and the kidneys. Also, it is noted that, while the expanded distal sections (206, 306, 406A, 406B) of a cannula are the most flexible and bendable sections, a collapsed distal section (106) also has more flexibility and bendability than the solid, no-hole portions of a cannula of a medical device according to the invention.

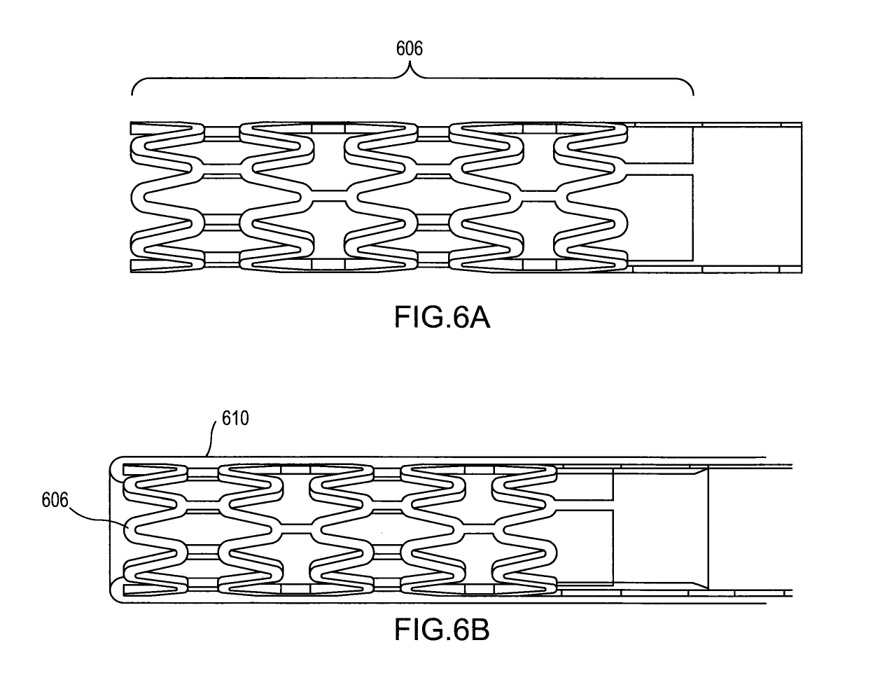

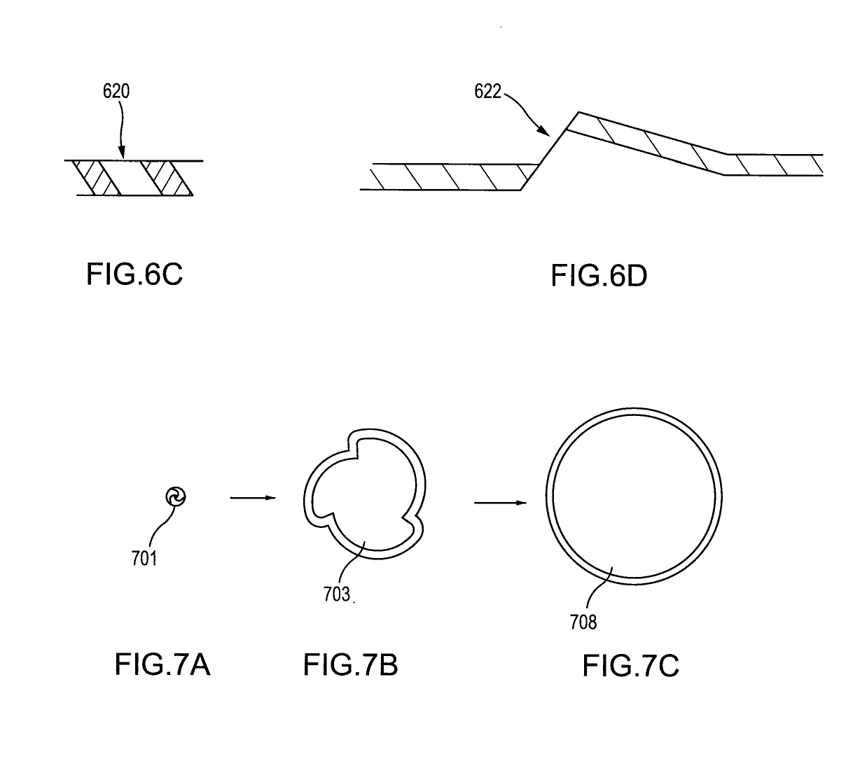

Whether a medical device (100, 200, 300, 400) of the invention comprises a catheter (104, 204, 304, 404) with one, two, three, or more flexible sections (106, 206, 306, 406A, 406B), each of these flexible sections includes a plurality of holes disposed in and around the entire wall of the section. The holes can be formed in any of a number of ways, such as by laser cutting into the wall of the cannula to form the cut section. The collection of holes in each section is intended to accomplish a number of functions including providing a more flexible section and allowing fluid to pass. The hole patterns that are formed are similar to that of a vascular stent, however the distal sections of the cannula with the holes are not intended to prop open vessel walls (as vascular stents are) but instead designed to provide flexibility and allow fluid flow therethrough. The shape, position, and geometry of the holes in any section of the cannula should be such that areas of stasis are minimized. Tapered wall thickness edges may be required to achieve the desired flow characteristics. As described in commonly-owned U.S. Patent Application Publication No. US 2007/0197856 A1 (which was published on Aug. 23, 2007, and which is incorporated herein by reference), tapered holes can provide a more deliberate sweeping of the fluid path to reduce thrombus formation. One possible configuration for each of the holes in the wall of the cannula can be as shown in FIG. 6C in which an angled hole 620 is shown formed in the cannula's wall, and another possibility of a hole configuration is shown in FIG. 6D in which a protruding side hole is formed in the cannula's wall. Each of the multiple protruding side holes can be open 622 as shown in FIG. 6D when the cannula is in its expanded state, and in the collapsed state the protrusion in the wall will retract to form a smooth and closed wall without any openings 622. The hole arrangement shown in FIG. 6A is possible and somewhat different than the hole arrangement shown in FIGS. 2B and 3B.

FIG. 6B shows the same distal section 606 of the expanded cannula as in FIG. 6A but with a flexible thin-walled sheath 610 disposed both inside and outside of at least this distal section 606 of the expanded cannula to cover the holes and thus allow the same flexibility and bendability of this distal section while maintaining a continuous fluid path through both the collapsed-diameter and expanded-diameter lumens of the device. This sheath 610 could extend for the entire length of the cannula or just one or more portions of it such as the distal section 606. And the sheath 610 could be just on the outside or just on the inside, instead of being both inside and outside. The sheath 610 can be polyurethane, vinyl, thermoplastic rubber, or a thermoset material like silicone, for example. One preferred material is urethane. The sheath 610 can be created by dipping or coating the cannula with liquid polymer, spray molded, blown film, extruded, injection molded, pultruded, or assembled from flat stock as a separate component, for example. Non-covered segments of the cannula will provide flow into or out of the cannula through any uncovered holes and through the distal opening of the through-lumen of the cannula.

Regarding drainage, it is noted that the length of the cut pattern can be a function of the minimum desired fluid flow, desired flow characteristics, and desired resultant fluid shear. This pattern will permit radial expansion of the cannula with minimal loss in length—to maintain desired placement during the expansion activity.

The cut pattern also enables the cannula to be more flexible and less prone to kinking. The patency of the lumen is critical when aspirating or returning blood. To access the pulmonary artery, the lumen cross-section should not reduce by any appreciable means. In order to access the pulmonary artery, flexibility of the portion of the cannula body that transitions to the heart and within it must maintain an open lumen. The cannula cuts may be dip coated with thin-walled polyurethane or other biocompatible material. The coating has a number of purposes including (i) to seal the lumens from draining blood through the cuts until it reaches the desired fluid ejection site (such as the pulmonary artery), (ii) to provide little resistance to the expansion of the cannula therefore requiring the material to stretch during the recovery of the cannula during expansion, and (iii) to coat the entire cannula, inside and out if required, to improve biocompatibility of the cannula.

Other possibilities for any of the holes include a circular shape, an oval shape, or any other shape. One or more of the holes could be flared in its expanded state to perform like a funnel in fluid aspiration.

Any collapsed-state cannula according to the invention should have the ability to recover (that is, expand) to its full diameter with minimal foreshortening and in a predictable manner from initial cannula placement in the collapsed state. To best accomplish this, the body of the cannula can be preformed with two or more two wings, folds, or lobes. The lobes will be placed about the cannula in the pre-expanded state and will migrate from folded to circular upon cannula expansion. The lobes may be uniform or non-symmetrical. FIGS. 7A, 7B, and 7C depict cross-sectional views of such a multi-lobed cannula embodiment of a medical device according to the invention, where the device can be any of the devices shown in FIGS. 1A, 2A, 3A, 4, and 5. FIGS. 7A and 7C show the cannula in its fully collapsed and fully expanded states, respectively. FIG. 7B shows the cannula in transition from the collapsed state to the expanded state. Axial folds of this multi-lobed cannula allow folding of the wall of the cannula as it reduces down to its collapsed state and unfolding of the wall of the cannula as it opens up to its expanded state, and these folds can be created by rolling the body of cannula. A lobe-forming tool, similar to those utilized to fabricate folded/expandable medical balloons may be utilized throughout the length of, or for a portion of the length of, the cannula, thereby making a small diameter for at least the access portion of the cannula. As the hub is approached, the cannula can transition from winged to circular. The body of the cannula diameter is reduced in axial cross section by such wing or lobe forming technique in which the material shape and wrapping the form axially compresses the overall diameter. This can be accomplished by drawing the part into a die that transitions the round cross section to one that permits wrapping. The resultant inside diameter results in an effective axial lumen to permit passage and guidance over a guidewire or catheter for advancing the cannula through the vasculature of a patient.

Referring now to FIG. 5, the medical device of FIG. 1A can be used with a peel sheath 500. The peel sheath 500 is used to control the expansion time of a temperature-activated SMP material of which the cannula or a portion of it is formed. Should the cannula 104 require a longer dwell time during placement, the peel sheath 500 is used to contain the collapsed cannula 104. This control sheath 500 can have the depicted peel-away configuration that allows the cannula to be advanced. Upon desired expansion, the sheath 500 is slid back towards the hub 102, fully exposing the cannula 104 to a warmed fluid that the operator introduces and/or to the warm fluid(s) and/or temperature inside the patient's body. The sheath 500 is separated into two halves as it is pulled from the patient's body and thus from around the cannula. The cannula remains in the patient's body. Sheaths such as the peel-away sheath 500 are known generally. The peel-away sheath 500 has two side tabs that can be grasped by the hands of an operator and pulled apart to peel the sheath in half along its length. A peelable sheath typically is scored along its length to facilitate peeling by an operator. Alternatively, a linear oriented polymer (molecular alignment in processing) can be utilized for a peel sheath (e.g., linear Teflon, linear polyethylene, etc.). Upon successful cannula placement and removal of the sheath 500, the extracorporeal circuit is completed, any clamps are removed from the proximal end of the cannula and/or the hub and/or the one or more tubes connected to the hub, and circulation is initiated through the expanded lumen of the device.

In a preferred embodiment, a cannula of a medical device according to the invention is constructed from a single SMP material. That SMP material is produced to the desired final diameter. The cannula is shaped to the desired two-dimensional or three-dimensional shape, and the cannula is then machined or laser cut to incorporate any desired hole pattern(s) of the expanded shape. The hub is then attached by, for example, insert molding or mechanical assembly, and a urethane membrane is applied to the desired target areas of the medical device.

In one particular embodiment of a medical device according to the invention, the compressed or collapsed diameter of the cannula could range from 0.06″ to 0.15″ with the target diameter being 0.08″. The wall thickness of the cannula is a function of the desired hoop strength of the polymer in the expanded state, and the wall thickness could range from 0.005″ to 0.050″. The cannula French size can vary from 8 F to 36 F but the concept can also be applied to Gage size catheters as used for IV infusion or other applications where placement is facilitated by being small but maximum fluid flow is desirable after placement. The inside diameter of the lumen of the medical device need not be consistent in that the tip of the cannula could have a smaller inside diameter and along the length of the cannula the inside diameter could increase to reduce fluid pressure drop. The cannula can have other features. For example, the portion of the cannula that emerges from the patient's body can be contained within a velour-like tube to enable tissue in-growth of the skin and reduce bacterial wicking about the wound. The velour material may be knitted or woven polyester or other textured material. A knitted material structure will expand and contract more easily than a woven structure and would be preferred at the skin site. The velour tube should be bonded to the outside diameter of the conduit. The lumen of the cannula can be larger than the blood vessel it is positioned in as it will only expand to the maximum allowable diameter. (Other materials or more elaborate designs may be used to encourage tissue ingrowth for the percutaneous access portion of the catheter) Alternatively, the distal end(s) of the cannula can be bell-mouthed to reduce aspiration tip shear or jet discharge. Depth placement for positioning of the proximal end can be identified by marker bands or a sliding ring band about the cannula. Radiopaque markers can be incorporated into the body of the cannula to aid in identifying the position of the cannula with radiography.

Regarding activation of the SMP material(s) used to form the cannula or one or more portions of the cannula, the trigger to cause the cannula to change from its collapsed state to its expanded state can be one or more of temperature, electricity, light, and moisture. A moisture-activated shape memory polymer changes its shape when exposed to water, a surgical solution, and/or blood. Temperature is the preferred trigger.

The SMP may require at least a temporary increase in temperature exposure over the application temperature to set the working shape of the polymer (T_{g}). For example, should the working temperature be 37° C. (body temperature), the material set temperature may be tailored to have a T_{g} (glass transition temperature) equal to 39 or 40° C. This could just as well be 100° C. (Tg), by the way, but the closer to body temperature, the quicker the response time. The material rise to body temperature after insertion will initiate the shape recovery (that is, expansion of the cannula). To expedite the shape return, heated saline may be injected through the cannula provided the fluid is below blood damage levels (41.5° C.). Once stabilized, the natural body temperature of 37° C. should provide a stable environment for functional use of the device. To slow the recovery, should that be required, the cannula can be collapsed and constrained in a support tube, such as the peel sheath 500 of FIG. 5, for placement into the body for expansion. This will insulate the polymer until ready for activation. After the sheath is removed, the material will recover to the desired expanded shape.

Also, the expanded catheter may be triggered to collapse while it is still placed within the body of the patient. For example, chilled saline may be passed into the catheter to cause it to collapse from its expanded shape in order to facilitate its removal from the patient's body. As indicated elsewhere herein, a trigger other than temperature is possible and can be used to expand and collapse the catheter repeatedly.

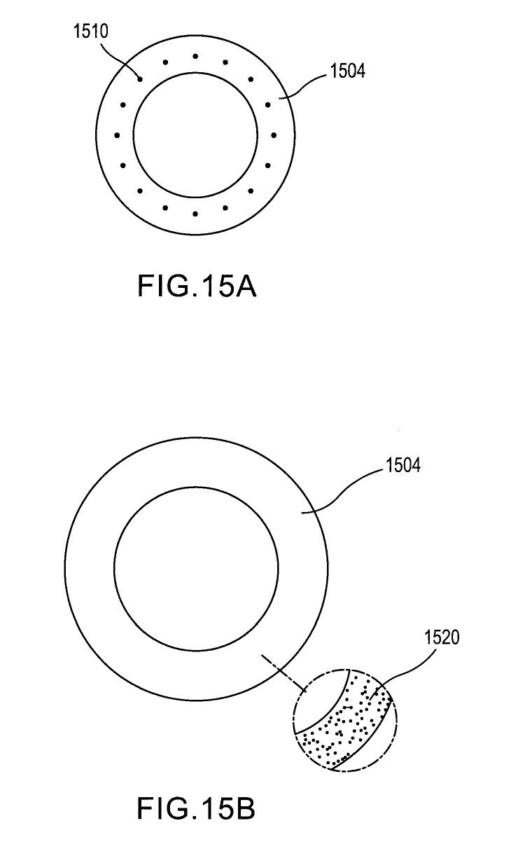

As indicated in FIG. 15A, the SMP can be fabricated with wires 1510 placed within the SMP wall of the cannula 1504, and resistance heating of these wires 1510 can be employed to induce a temperature change within the polymer to recover the SMP's desired shape. Alternatively, and as indicated in FIG. 15B, the SMP wall of the cannula 1504 may be filled with or otherwise made to contain a sufficient amount of an inductively sensitive material 1520 including, for example, something magnetic such as silica-coated magnetic nanoparticles of iron oxide, and then an internal induction coil can be placed within a cannula introducer and slid through the inside diameter of the cannula to activate the SMP as the induction coil passes by.

Light activation of an SMP also is possible. Still referring to FIG. 15B, the SMP wall of the cannula 1504 could be filled with or otherwise made to contain a sufficient amount of a laser-absorbing dye, and then an introducer/light guide can be utilized to expose the inside lumen of the cannula to diffused light for absorption into the dye to activate the SMP and cause the shape recovery of the cannula.

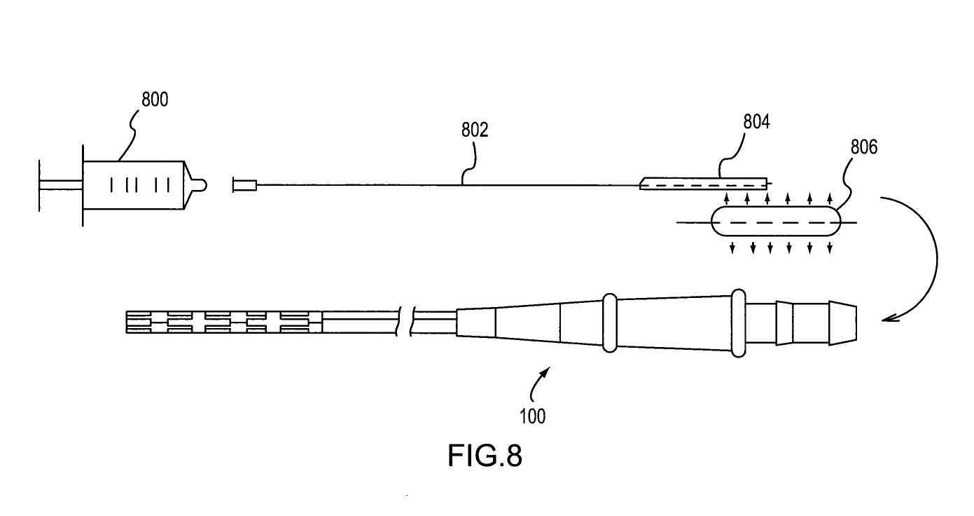

Turning now to FIG. 8, one alternative embodiment of a medical device does not use any SMP material at all. Instead, a device 100 similar to the one depicted in FIG. 1A has a distal section of its cannula configured to be mechanically expanded by a balloon. In this alternative embodiment of the device, an expandable balloon is used. The deflated balloon 804 is coupled to a syringe 800 which can provide fluid through a conduit 802 to fill the balloon and create an inflated balloon 806. With the balloon deflated, it is inserted into the lumen of the collapsed-state device 100, and then the syringe 800 is used to push fluid (such as saline) into the balloon to inflate the balloon which in turn mechanically pushes at least the collapsed distal section 106 into its expanded state.

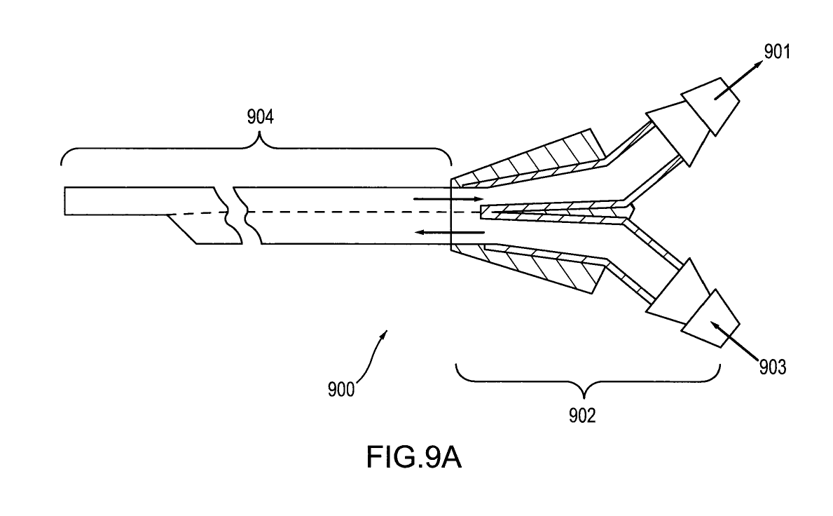

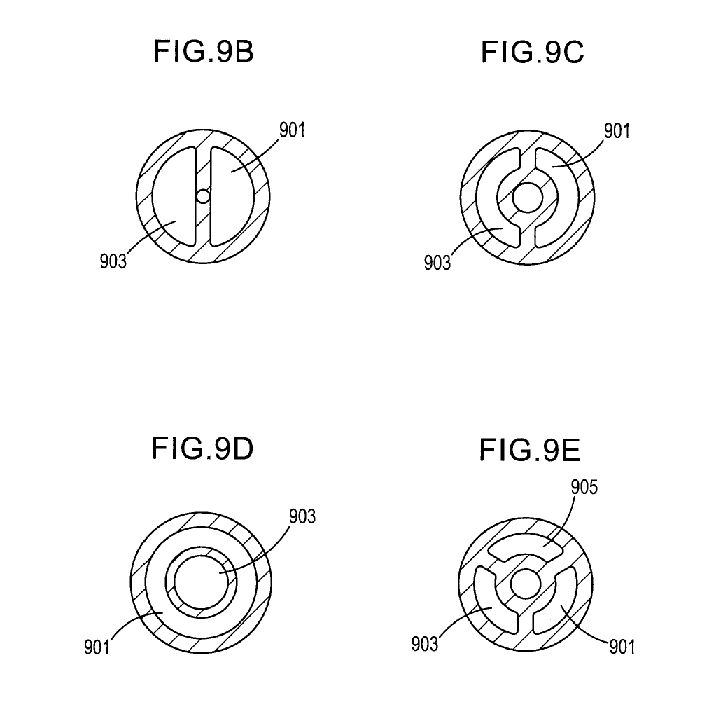

Turning back to embodiments according to the invention that use one or more SMP materials, reference is made to FIG. 9A which is a cross-sectional view of an embodiment of a medical device 900 according to the invention. The device contains at least two lumens 901, 903 within a cannula 904, as opposed to the previously-disclosed single-lumen embodiments. This dual-lumen device 900 of FIG. 9A will be advanced into the body of the patient in the collapsed state with the distal ends of each lumen positioned in different locations typically due to the distance between the distal tips of each of the two separate lumens. For a two-lumen cannula like depicted in FIG. 9A, one lumen could be positioned to perform as a drainage cannula, and the other lumen would then be a fluid return cannula. The distance between the two distal ends of the cannulae openings may be 10 cm or more apart. An example would be percutaneous cannulae placement through the neck with the outer lumen used for pump aspiration and located in the superior vena cava and the return lumen placed in or near the right atrium. When expanded, the cannulae will provide fluid conduits to allow blood to be removed and returned to the body. Either lumen may be drainage or return depending upon the application. Alternatively, both lumens could be used for drainage or return. As shown in FIGS. 9B-9D, the two lumens can be segmented within the cannula 904 or co-axial within the cannula 904. FIG. 9E shows that a medical device according to the invention can have three lumens in the cannula portion of the device. An optional center lumen can be provided for receiving a guidewire, as indicated in FIGS. 9B, 9C, and 9E. The inner co-axial lumen of FIG. 9D can receive a guidewire. Any lumen used to receive a guidewire does not necessarily need to be centered. One or more of the lumens defined by a dual- or multi-lumen device according to the invention can have one or more of the flexible sections described herein and/or one or more folded sections as also described herein.

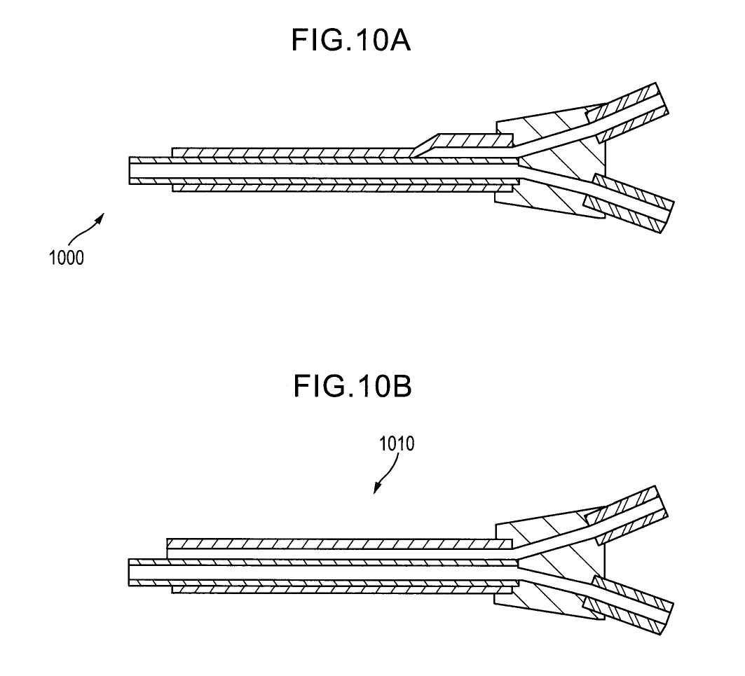

As shown in FIGS. 10A and 10B, another embodiment according to the invention involves a dual-lumen cannula with one lumen that maintains a single desired maximum diameter while the other lumen starts in its collapsed (that is, small diameter) form. This is shown by the device 1000 of FIG. 10A. Only the collapsed lumen need be made from the SMP material(s). The rationale for keeping one of the lumens fabricated in the fully dilated (that is, expanded) state is to use this portion of the cannula as the introducer. Maintaining the second lumen in the collapsed state during insertion into the body of the patient allows the cannula diameter to remain small, thus facilitating ease of cannulation. Expanding the second lumen upon successful insertion into the final position within the patient's body allows increased flow capability of the circuit to enhance circulatory or respiratory support. The placed and expanded device 1010 is shown in FIG. 10B.

With all of these dual-lumen embodiments (as shown, for example, in FIGS. 9A, 10A, 10B, 11A, and 11B) the two lumens split in the hub of the device and transition into two single lumens, each with a port for connection to the extracorporeal circuit. It is primarily or exclusively the implanted (into the patient's body) portion of the cannula that is collapsed to enable ease of vascular access as described herein. The collapsed lumen may be wing- or lobe-formed as described previously, and the collapsed lumen can include hole patterns for drainage and return fluid flow. In addition, one or both lumens can be pre-shaped for ease of distal tip placement within the vessel of the patient's body.

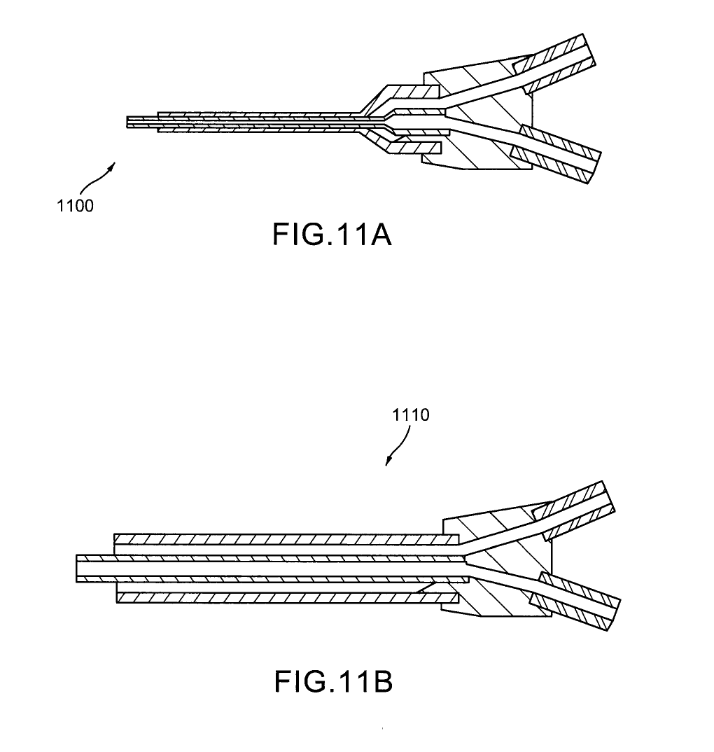

As shown in FIGS. 11A and 11B, another embodiment of a medical device according to the invention involves a dual-lumen cannula with inner and outer lumens that are both expandable. The device 1100 of FIG. 11A is shown in the collapsed state with an inside diameter and an outside diameter of both the inner and outer coaxial lumens minimized. In FIG. 11A, the collapsed outer lumen prevents flow therethrough. The device 1110 of FIG. 11B is the device 1100 but in its expanded state where both of the coaxial lumens are expanded and in particular the outer coaxial lumen is expanded to allow flow therethrough.

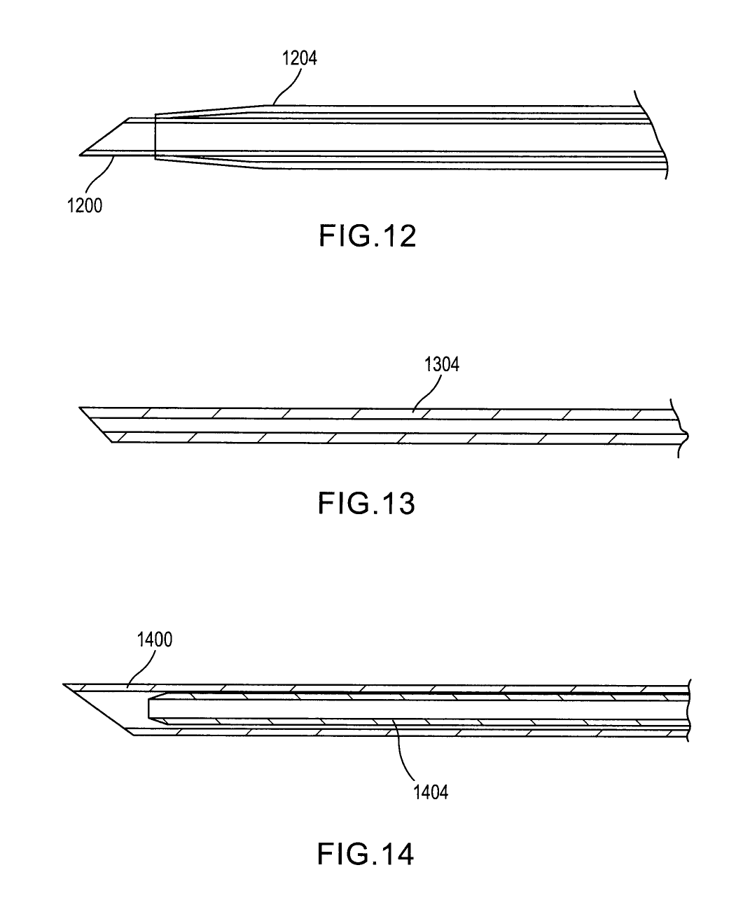

Tubular shaped catheters as described herein may provide one-way fluid delivery (to the body of a patient) not necessarily combined into a recirculation circuit or system. As such, the use of SMP material(s) in catheter applications such as intravenous (IV) catheters may be placed over or within an introducer/puncture needle to gain vena-puncture. Such a device would permit small diameter access but expand to enable higher flow volumes for rapid infusion of blood, saline, or medical solutions. A smaller catheter may enable access into difficult vena-puncture patients but permit more or maximum fluid introduction than normally attained by standard technology. Alternatively, the SMP material(s) may be sharpened at the tip to enable direct puncture of tissue without requiring an introducer needle. Tubular shaped ports as those utilized for laparoscopic or endoscopic access may be introduced that will expand to enable smaller puncture sites but permit larger devices to access the body cavity once the port is expanded. FIG. 12 is a cross-sectional view of a collapsed-state/non-expanded cannula 1204 (such as or similar to the cannula 104 shown in side view in FIG. 1A) with a distal end portion that tapers down and with an introducer needle 1200 extending through the lumen of the cannula 1204. FIG. 13 is a cross-sectional view of a collapsed-state/non-expanded cannula 1304 with a sharpened and angled distal end to allow it to puncture the skin or other tissue of a patient without the need for a separate introducer needle. And FIG. 14 is a cross-sectional view of a collapsed-state/non-expanded cannula 1404 (such as or similar to the cannula 104 shown in side view in FIG. 1A) disposed within a lumen of an introducer needle 1400.

Shape memory polymers generally are known but will be described to some degree herein for completeness. Most SMP materials can retain two shapes, and the transition between those is usually induced by temperature. In addition to temperature change, the shape change of SMPs can also be triggered by an electric, or magnetic field, light, humidity, or solution. As well as polymers in general, SMPs also cover a wide property-range from stable to biodegradable, from soft to hard, and from elastic to rigid, depending on the structural units that constitute the SMP. SMPs include thermoplastic and thermoset (covalently cross-linked) polymeric materials. SMPs are known to be able to store up to three different shapes in memory. Two important quantities that are used to describe shape memory effects of SMPs are the strain recovery rate (Rᵣ) and strain fixity rate (R_{f}). The strain recovery rate describes the ability of the material to memorize its permanent shape, while the strain fixity rate describes the ability of switching segments to fix the mechanical deformation. Polymers exhibiting a shape memory effect have both a temporary form and a stored (permanent) form. Once the latter has been manufactured by conventional methods, the material is changed into another temporary form by processing through heating, deformation, and finally, cooling. The polymer maintains this temporary shape until the shape change into the permanent form is activated by a predetermined external stimulus. The shape change capability of these materials lies in their molecular network structure, which contains at least two separate phases. The phase showing the highest thermal transition, Tₚₑᵣₘ, is the temperature that must be exceeded to establish the physical crosslinks responsible for the permanent shape. The switching segments, on the other hand, are the segments with the ability to soften past a certain transition temperature (Tₜᵣₐₙₛ) and are responsible for the temporary shape. In some cases, this is the glass transition temperature (T_{g}) and others the melting temperature (Tₘ). Exceeding Tₜᵣₐₙₛ (while remaining below Tₚₑᵣₘ) activates the switching by softening these switching segments and thereby allowing the material to resume its original (permanent) form. Below Tₜᵣₐₙₛ, flexibility of the segments is at least partly limited. If Tₘ is chosen for programming the SMP, strain-induced crystallization of the switching segment can be initiated when it is stretched above Tₘ, and subsequently cooled below Tₘ. These crystallites form covalent netpoints which prevent the polymer from reforming its usual coiled structure. The hard to soft segment ratio is often between 5/95 and 95/5, but ideally this ratio is between 20/80 and 80/20. The shape memory polymers are effectively viscoelastic and many models and analysis methods exist.

Light activated shape memory polymers (LASMP) use processes of photo-crosslinking and photo-cleaving to change T_{g}. Photo-crosslinking is achieved by using one wavelength of light, while a second wavelength of light reversibly cleaves the photo-crosslinked bonds. The effect achieved is that the material may be reversibly switched between an elastomer and a rigid polymer. Light does not change the temperature, only the cross-linking density within the material. For example, it has been reported that polymers containing cinnamic groups can be fixed into predetermined shapes by UV light illumination (>260 nm) and then recover their original shape when exposed to UV light of a different wavelength (<260 nm). Examples of photoresponsive switches include cinnamic acid and cinnamylidene acetic acid.

The use of electricity to activate the shape memory effect of polymers is desirable for applications where it would not be possible to use heat. Conducting SMP composites with carbon nanotubes can be used such as short carbon fibers (SCFs), carbon black, and metallic Ni powder. These conducting SMPs are produced by chemically surface-modifying multi-walled carbon nanotubes (MWNTs) in a mixed solvent of nitric acid and sulfuric acid, with the purpose of improving the interfacial bonding between the polymers and the conductive fillers. The shape memory effect in these types of SMPs have been shown to be dependent on the filler content and the degree of surface modification of the MWNTs, with the surface modified versions exhibiting good energy conversion efficiency and improved mechanical properties. Also, surface-modified super-paramagnetic nanoparticles can be used. When introduced into the polymer matrix, remote actuation of shape transitions is possible. An example of this involves the use of oligo (e-caprolactone) dimethacrylate/butyl acrylate composite with between 2-12% magnetite nanoparticles. Nickel and hybrid fibers can also be used.

Certain embodiments according to the invention have been disclosed. These embodiments are illustrative of, and not limiting on, the invention. Other embodiments, as well as various modifications and combinations of the disclosed embodiments, are possible and within the scope of the disclosure.

What is claimed is:

1. A catheter with a collapsed diameter sufficient to allow placement of the catheter over a guidewire and into a body of a patient and being expandable in diameter after placement in the body of the patient to support fluid flow at a predetermined rate higher than a rate possible with the collapsed diameter, the catheter comprising:
an elongated member including a tubular wall defining at least one lumen extending therethrough, the elongated member comprising a shape memory polymer which allows at least a portion of the elongated member to expand its diameter beyond its collapsed diameter when exposed to at least one triggering stimulus, at least one section of the elongated member comprising a plurality of holes in the tubular wall to make that section more flexible and bendable than other parts of the elongated member without such holes.

2. A medical device for insertion into a blood vessel of a patient and for carrying one or more fluids into and/or out of the patient's body, the medical device comprising:
a hub for placement outside the patient's body; and
an elongated member extending from the hub and defining at least one passageway, at least one portion of the elongated member comprising a shape memory polymer and for placement within the patient's blood vessel when the elongated member is in a compressed state in which the at least one passageway is sized to receive a guidewire but not to carry fluid at a sufficiently high predetermined rate into and/or out of the patient's body, the at least one portion of the elongated member configured to change to an expanded state after placement within the patient's blood vessel in order to be able to carry fluid at the sufficiently high predetermined rate, at least one section of the at least one portion of the elongated member being more flexible than at least the remainder of the at least one portion of the elongated member.

3. The medical device of claim 2 wherein the at least one section of the at least one portion of the elongated member comprises a plurality of holes in a wall of the elongated member to make that section more flexible and bendable than other parts of the elongated member without such holes.

4. The medical device of claim 2 wherein the elongated member further comprises at least one other section with a plurality of axial folds that unfold when the elongated member is in the expanded state.

5. The medical device of claim 2 wherein the blood vessel is a vein or an artery and wherein at least blood is carried into and/or out of the patient's body.

6. The medical device of claim 2 wherein the elongated member defines two passageways.

7. The medical device of claim 2 wherein the shape memory polymer is activated by at least one of heat, light, and electricity in order to change the elongated member from the compressed state to the expanded state.

8. The medical device of claim 2 wherein the elongated member is configured such that it does not exert a pushing force on the interior wall of the patient's blood vessel when placed within the patient's blood vessel and in the expanded state.

9. The medical device of claim 8 wherein the placed and expanded elongated member does not block the flow of blood through the blood vessel but instead allows blood to flow between the interior wall of the patient's blood vessel and the exterior surface of the elongated member.

10. The medical device of claim 2 wherein the hub includes connectors for connecting to an extracorporeal support system.

11. A medical device for insertion into a blood vessel of a patient and for carrying one or more fluids into and/or out of the patient's body, the medical device comprising:
an elongated member defining at least one passageway, at least one portion of the elongated member comprising a shape memory polymer and for placement within the patient's blood vessel when the elongated member is in a compressed state in which the at least one passageway is sized to receive a guidewire but not to carry fluid at a sufficiently high predetermined rate into and/or out of the patient's body, the at least one portion of the elongated member configured to change to an expanded state after placement within the patient's blood vessel in order to be able to carry fluid at the sufficiently high predetermined rate, at least one section of the at least one portion of the elongated member comprising a plurality of axial folds that unfold when the elongated member is in the expanded state.

12. The medical device of claim 11 wherein the elongated member further comprises at least one other section that comprises a plurality of holes in a wall of the elongated member to make that section more flexible and bendable than other parts of the elongated member without such holes.

13. The medical device of claim 11 wherein the blood vessel is a vein or an artery.

14. The medical device of claim 13 wherein at least blood is carried into and/or out of the patient's body.

15. The medical device of claim 11 wherein the elongated member defines two passageways.

16. The medical device of claim 15 wherein the two passageways are oriented coaxially or side-by-side.

17. The medical device of claim 11 wherein the shape memory polymer is activated by at least one of heat, light, and electricity in order to change the elongated member from the compressed state to the expanded state.

18. The medical device of claim 11 wherein the elongated member is configured such that it does not exert a pushing force on the interior wall of the patient's blood vessel when placed within the patient's blood vessel and in the expanded state.

19. The medical device of claim 18 wherein the placed and expanded elongated member does not block the flow of blood through the blood vessel but instead allows blood to flow between the interior wall of the patient's blood vessel and the exterior surface of the elongated member.

20. The medical device of claim 11 further comprising a hub from which the elongated member extends, the hub including connectors for connecting to an extracorporeal support system.
